(19) United States
(12) Patent Application Publication
(10) Pub. No.: US 2024/0197875 A1
(43) Pub. Date: Jun. 20, 2024

(54) NOVEL METHOD

(71) Applicant: GAMMADELTA THERAPEUTICS LIMITED, London (GB)

(72) Inventors: Andrew John HUTTON, London (GB); Istvan KOVACS, London (GB); Oliver NUSSBAUMER, London (GB)

(73) Assignee: GAMMADELTA THERAPEUTICS LIMITED, London (GB)

(21) Appl. No.: 18/554,501

(22) PCT Filed: Apr. 8, 2022
(86) PCT No.: PCT/GB2022/050886

§ 371 Date: Oct. 9, 2023

(30) Foreign Application Priority Data
Apr. 9, 2021 (GB) 2105113.1

(51) Int. Cl. A61K 39/00; C12N 5/0783

(52) U.S. Cl. CPC A61K 39/4631; A61K 39/4611; A61K 39/464468; C12N 5/0636; C12N 2501/2315; C12N 2501/2321; C12N 2502/1114; C12N 2510/00

(57) ABSTRACT

The invention relates to methods for expanding γδ T cells comprising preparing a composition enriched for γδ T cells and culturing the composition in the presence of feeder cells. Also provided is a method for engineering γδ T cells comprising preparing a composition enriched for γδ T cells, transducing the composition to express a chimeric antigen receptor (CAR) specific for a tumour associated antigen and culturing the transduced composition to expand the engineered γδ T cells. Also provided are expanded and engineered γδ T cells produced according to the described methods, which find utility in adoptive T cell therapies, chimeric receptor therapies and the like.

### FIELD OF THE INVENTION

The invention relates to methods for expanding γδ T cells, said method comprising the steps of preparing a composition enriched for γδ T cells and culturing said composition in the presence of feeder cells. Also provided is a method for engineering γδ T cells, said method comprising the steps of preparing a composition enriched for γδ T cells, transducing the composition to express a chimeric antigen receptor (CAR) specific for a tumour associated antigen and culturing the transduced composition to expand the engineered γδ T cells. Such γδ T cells include non-Vδ2 cells, e.g. Vδ1, Vδ3 and Vδ5 cells. Expanded and engineered γδ T cells produced according to the methods described herein find utility in adoptive T cell therapies, chimeric receptor therapies and the like. The present invention also relates to both individual cells and populations of cells produced by the methods described herein.

### BACKGROUND OF THE INVENTION

The growing interest in T cell immunotherapy for cancer has focused on the evident capacity of subsets of CD8⁺ and CD4⁺αβ T cells to recognise cancer cells and to mediate host-protective functional potentials, particularly when de-repressed by clinically mediated antagonism of inhibitory pathways exerted by PD-1, CTLA-4, and other receptors. However, αβ T cells are MHC-restricted, which can lead to graft versus host disease in an allogeneic setting.

The treatment of cancer with adoptive cell therapy is largely limited to platforms based on circulating, patient-derived, engineered autologous αβ T cells. Although successful in some haematological malignancies, this approach comes with challenges including associated toxicities, high production costs and a requirement to gene edit cells to avoid graft vs host disease if used in an allogeneic setting. While engineered αβ T cells have shown therapeutic activity in haematological malignancies, clinical activity in solid tumours has been challenging.

Gamma delta T cells (γδ T cells) represent a subset of T cells that express on their surface a distinct, defining γδ T-cell receptor (TCR). This TCR is made up of one gamma (γ) and one delta (δ) chain. Human γδ TCR chains are selected from three main 0 chains, Vδ1, Vδ2 and Vo3 and six γ chains. Human γδ T cells can be broadly classified based on their TCR chains, as certain γ and δ types are found on cells more prevalently, though not exclusively, in one or more tissue types. For example, most blood-resident γδ T cells express a Vδ2 TCR, for example Vγ9Vδ2, whereas this is less common among tissue-resident γδ T cells, which more frequently use Vδ1 in skin and Vγ4 in the gut.

Thus, in contrast to αβ T cells, Vo1 γδ T cells are a subset of innate T cells defined by expression of T cell receptors composed of a γ chain paired to a Vδ1 chain. In mice, Vδ1 γδ T cells are predominantly tissue resident where they are highly protective against a broad spectrum of carcinomas by mediating anti-tumour responses via pattern and natural cytotoxicity receptor recognition. Similarly, in humans, Vδ1 γδ T cells predominantly reside within epithelial tissues, mediate target cell recognition that is not MHC restricted and are not allo-HLA reactive. HLA matching of patients is therefore not required for γδ T cell adoptive cell therapies. The innate Vδ1 γδ T cell biology which enables antigen independent tumour recognition, lack of necessity for HLA matching, and inherent migration to and residence in human tissues makes Vδ1 γδ T cells an attractive platform for cellular therapy.

There is therefore a need for methods to efficiently expand γδ T cells to allow their adaptation as therapies, e.g. as adoptive T cell therapies, and for methods which have the potential to provide allogeneic ‘off-the-shelf’ chimeric antigen receptor-expressing γδ T cell therapies, such as for the treatment of solid tumours.

WO2017072367 and WO2018202808 relate to methods of expanding non-haematopoietic tissue-resident γδ T cells in vitro by culturing lymphocytes obtained from non-haematopoietic tissue in the presence of at least Interleukin-2 (IL-2) and/or Interleukin-15 (IL-15). WO2015189356 describes a composition for expanding lymphocytes obtained from a sample obtained by aphaeresis comprising at least two types of cytokines selected from IL-2, IL-15 and IL-21.

Therefore, while these disclosures go some way towards addressing the above-mentioned problem, there remains a need for methods of expanding and engineering γδ T cells, such as from skin, that provide the ability to use such γδ T cells in therapy.

### SUMMARY OF THE INVENTION

According to a first aspect of the invention, there is provided a method for expanding γδ T cells, wherein said method comprises the steps of:
(i) preparing a composition enriched for γδ T cells; and
(ii) culturing the composition in the presence of feeder cells, wherein the feeder cells are present in a ratio of at least 4:1 (feeder cells: γδ T cells).

According to another aspect of the invention, there is provided a method for expanding γδ T cells, wherein said method comprises the steps of:
(i) preparing a composition enriched for γδ T cells; and
(ii) culturing the composition in the presence of feeder cells and media comprising IL 15 and IL-21, wherein the feeder cells are present in a ratio of at least 3:2 (feeder cells: γδ T cells).

According to a further aspect of the invention, there is provided a method for expanding γδ T cells, wherein said method comprises the steps of:
(i) preparing a composition enriched for γδ T cells by depletion of αβ T cells; and
(ii) culturing the composition in the presence of feeder cells, wherein the feeder cells are present in a ratio of at least 3:2 (feeder cells: γδ T cells).

According to a yet further aspect of the invention, there is provided a method for engineering γδ T cells, said method comprising the steps of:

(i) preparing a composition enriched for γδ T cells;

(ii) transducing the composition to express a chimeric antigen receptor (CAR) recognizing a tumour antigen in the absence of TCR stimulation; and

(iii) culturing the transduced composition to expand the engineered γδ T cells, wherein steps (ii) and (iii) may be performed in either order or concurrently.

According to one aspect of the invention, there is provided an expanded γδ T cell population obtainable, such as obtained, by the methods described herein. According to a further aspect, there is provided an engineered γδ T cell population obtainable, such as obtained, by the methods described herein.

According to another aspect of the invention, there is provided a pharmaceutical composition comprising the expanded γδ T cell population or the engineered γδ T cell population as described herein.

According to a yet further aspect of the invention, there is provided the expanded γδ T cell population, the engineered γδ T cell population or the pharmaceutical composition as described herein for use as a medicament. In another aspect, there is provided the expanded γδ T cell population, the engineered γδ T cell population or the pharmaceutical composition as described herein for use in the treatment of cancer, such as such as for the treatment of solid tumours.

Also provided is a method for expanding γδ T cells, wherein said method comprises the steps of:
(i) preparing a composition enriched for γδ T cells; and
(ii) culturing the composition in the presence of feeder cells, wherein the feeder cells are present in a ratio of at least 1:2 (feeder cells: γδ T cells), especially at least 1:1 (feeder cells: γδ T cells), in particular at least 2:1 (feeder cells: γδ T cells), such as at least 3:1 (feeder cells: γδ T cells).

Further provided is a method for expanding γδ T cells, wherein said method comprises the steps of:
(i) preparing a composition enriched for γδ T cells; and
(ii) culturing the composition in the presence of feeder cells and media comprising IL-15 and IL-21, wherein the feeder cells are present in a ratio of at least 1:2 (feeder cells: γδ T cells), such as at least 1:1 (feeder cells: γδ T cells).

Additionally provided is a method for expanding γδ T cells, wherein said method comprises the steps of:
(i) preparing a composition enriched for γδ T cells by depletion of αβ T cells; and
(ii) culturing the composition in the presence of feeder cells, wherein the feeder cells are present in a ratio of at least 1:2 (feeder cells: γδ T cells), such as at least 1:1 (feeder cells: γδ T cells).

### BRIEF DESCRIPTION OF THE FIGURES

FIG. 1: Comparison of various sources of feeder cells for γδ T cell expansion. γδ T cells were isolated from skin, depleted of αβ T cells, and cocultured for 7 days with following irradiated cells: allogeneic peripheral blood lymphocytes (PBLs), allogeneic peripheral blood mononuclear cells (PBMCs), anti-CD3 CD28 activated allogeneic PBMCs (Act PBMCs) or allogeneic skin isolation cultures (Skin αβ/Skin iso cells), as compared to control (4CK). A) Proliferation is shown using the Ki67 marker, and total Vδ1 cell number is shown in B.

FIG. 2: Expansion of γδ cells when γδ T cells are positively selected out from initial population and added back to the remaining population of feeder cells in various proportions. A) The fold expansion of γδ T cells was determined after the indicated days in culture in the presence of feeder cells (1%, 5%, 10%, 20%, or 40% γδ T cell enriched, non-αβ cell content at DO with the remainder of the culture made up of autologous feeder cells) following positive selection for the γδ T cells. B) As for A in the absence of feeder cells.

FIG. 3: Expansion of γδ cells when αβ cells are depleted from initial population and added back as feeder cells in various proportions. A) The fold expansion of γδ T cells was determined after the indicated days in culture in the presence of feeder cells (1%, 5%, 10%, 20%, or 40% non-αβ cell content at DO with the remainder of the culture made up of autologous feeder cells). B) As for A in the absence of feeder cells.

FIG. 4: CD19 CAR⁺ γδ T cells display cytotoxic activity against NALM6 target cells. Compositions of γδ T cells including αβ feeder cells were transduced with CD19 CAR followed by the removal of feeder cells from the expanded γδ T cells by depletion of the αβ T cells. The transduced γδ T cells were then incubated with NALM6 target cells expressing CD19 at the indicated ratios and the amount of killing measured.

FIG. 5: Cryopreserved transduced γδ T cells are viable, CAR expression is stable and cytotoxic activity is retained post-thaw. A) CAR expression levels on day 14 of expansion, before freezing. B) As for A, the proportion of CD19 CAR⁺ cells was measured seven days post-thaw, demonstrating good levels of expression even after freezing and recovery.

FIG. 6: Post-thaw CD19 CAR transduced γδ T cells, wherein the feeder cells were removed by depletion of αβ T cells, were incubated with NALM6 target cells expressing CD19 at the indicated ratios and the amount of killing measured.

FIG. 7: Meso-CAR transduced γδ T cells, wherein the starting γδ T population were αβ depleted, then transduced and cultured as described, resulting in a γδ T cell population that is more than 40% mesothelin CAR⁺.

FIG. 8: γδ T cells transduced with a meso-CAR display cytotoxicity against mesothelin positive cancer cell lines. A) Viability of mock and transduced cells post-thaw B) Cytotoxicity vs Hela cells C) Cytotoxicity vs SCOV-3 cells.

FIG. 9: Negatively selected γδ T cells were cultured with either skin resident CD4 αβ T cells (“CD4 Feeder”), skin resident CD8 αβ T cells (“CD8 Feeder”), both CD4 and CD8 αβ T cells (“αβ Feeder”) or alternatively cultured with no additional feeder cells added (“γδ only”). Cultures were then expanded for either 14 days (left graph) or 21 days (right graph) before cultures were harvested and γδ expansion rate calculated.

### DETAILED DESCRIPTION OF THE INVENTION

It has been previously reported that populations of γδ T cells can be expanded to a clinical scale using irradiated artificial antigen presenting cells (aAPC) as feeders (Deniger et al., Clin. Cancer Res., 2014; 20(22): 5708-5719). Such aAPC are derived from K562 tumour cells and express CD137L which, in the presence of IL-2 and IL-21, leads to the activation and propagation of a polyclonal γδ T cell population. However, such methods require the genetic modification of K562 tumour cells in order to them to function as aAPC and support γδ T cell expansion and activation, as well as irradiation to arrest the growth of these tumour derived aAPC.

Therefore, according to a first aspect of the invention, there is provided a method for expanding γδ T cells, wherein said method comprises the steps of:
(i) preparing a composition enriched for γδ T cells; and
(ii) culturing the composition in the presence of feeder cells, wherein the feeder cells are present in a ratio of at least 4:1 (feeder cells: γδ T cells).

The methods described herein are performed outside the human or animal body, i.e. they are in vitro and/or ex vivo. Thus, in one embodiment the methods described herein are in vitro methods. In a further embodiment, the methods described herein are ex vivo methods.

As used herein, references to “expanded”, “expanded population” or expanded γδ T cells” includes populations of cells which are larger or contain a larger number of cells than a non-expanded population. Such populations may be large in number, small in number or a mixed population with the expansion of a proportion or particular cell type within the population. It will be appreciated that the term “expansion step” refers to processes which result in expansion or an expanded population. Thus, expansion or an expanded population may be larger in number or contain a larger number of cells compared to a population which has not had an expansion step performed or prior to any expansion step. It will be further appreciated that any numbers indicated herein to indicate expansion (e.g. fold-increase or fold-expansion) are illustrative of an increase in the number or size of a population of cells or the number of cells and are indicative of the amount of expansion.

It will be appreciated that culturing the composition of γδ T cells is performed for a duration of time effective to produce an expanded population of γδ T cells. In one embodiment, a duration of time effective to produce an expanded population of γδ T cells is at least 7 days. Thus, in one embodiment, the composition of γδ T cells is cultured for at least 7 days. In a further embodiment, the composition is cultured for between 7 and 21 days, such as 9 to 15 days. In yet further embodiments, the composition is cultured for about 10, 11, 12, 13 or 14 days.

In still further embodiments, the composition is cultured for at least 5 days, at least 6 days, at least 7 days, at least 8 days, at least 9 days, at least 10 days, at least 11 days, at least 12 days, at least 13 days, at least 14 days or at least 21 days, e.g. about 14 days or about 21 days to produce an expanded population of γδ T cells. In one embodiment, the composition is cultured for about 10, 11, 12, 13 or 14 days to produce an expanded population of γδ T cells.

Suitably expanding the population of γδ T cells provides at least a 5-fold, especially at least a 10-fold, in particular at least a 20-fold, such as at least a 50-fold, for example at least a 100-fold number of γδ T cells.

In one embodiment, the method comprises freezing the expanded γδ T cells. Such frozen expanded γδ T cells may subsequently be thawed for downstream processing or use, such as therapeutic use. Freezing allows the easy transport and long-term storage of expanded γδ T cells and is well known in the art. Therefore, a method that provides for cells that show good viability and activity after freezing and thawing is advantageous, and not all expansion methods yield such cells (data not shown).

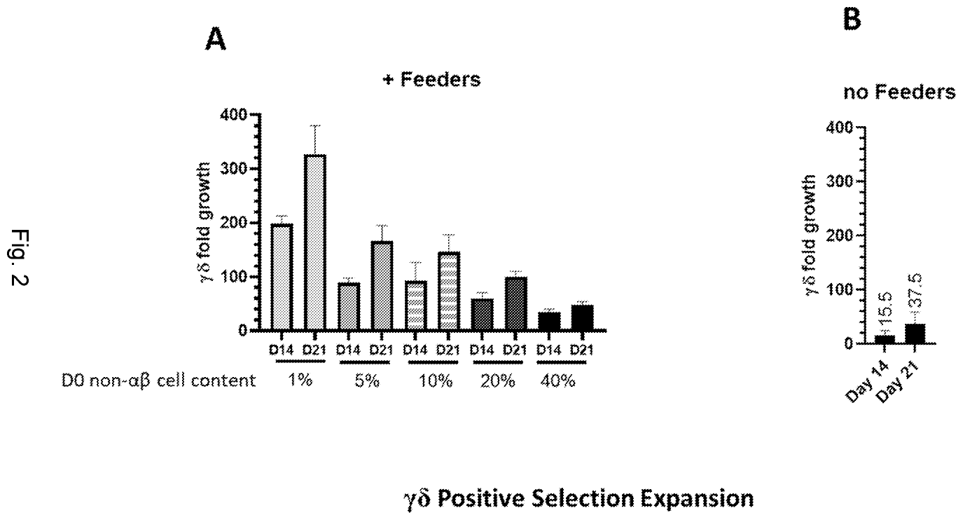

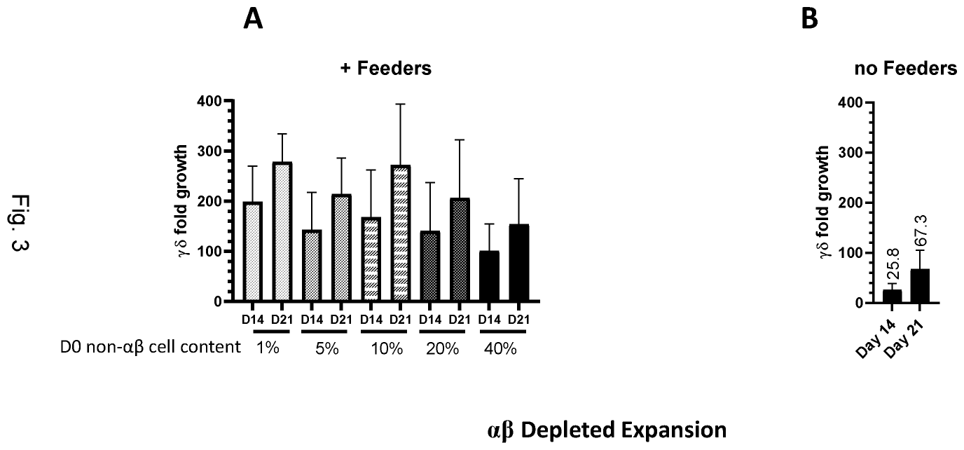

A feeder cells: γδ T cells ratio of at least 4:1 is equal to a proportion of at least 80% feeder cells to 20% or fewer γδ T cells in the culture. Such a ratio of feeder cells: γδ T cells yields greatly enhanced expansion of the γδ T cell population in culture compared to a γδ T cell population cultured in the absence of feeder cells (FIGS. 2 to 3). Furthermore, the purity of γδ T cells in the CD45⁺ population within these cultures is increased even at the highest levels of feeder cell addition (data not shown). These advantageous effects are seen at all time points in culture, particularly at day 14 and day 21 of culture.

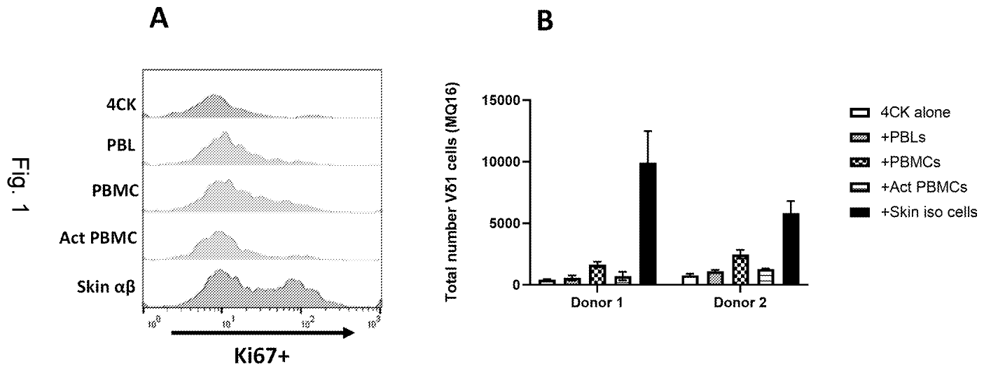

Thus, in one embodiment the culture comprises at least 80% feeder cells. In some embodiments, the feeder cells are present in a ratio of about 10:1 to about 99:1 (feeder cells: γδ T cells). In one embodiment, the feeder cells are present in a ratio of at least 10:1 (feeder cells: γδ T cells). Thus, in a further embodiment the culture comprises at least 90% feeder cells. In a further embodiment, the feeder cells are present in a ratio of at least 20:1 (feeder cells: γδ T cells). Thus, according to one embodiment the culture comprises at least 95% feeder cells. In a yet further embodiment, the feeder cells are present in a ratio of at least 50:1 (feeder cells: γδ T cells). Thus, in one embodiment the culture comprises at least 98% feeder cells. In a still further embodiment, the feeder cells are present in a ratio of at least 99:1 (feeder cells: γδ T cells). Thus, in a further embodiment the culture comprises at least 99% feeder cells. All ratios tested herein provide greatly enhanced expansion of the γδ T cell population in culture compared to a γδ T cell population cultured without feeder cells (FIG. 1), with particularly good yield and purity of γδ T cells when the feeder cells are present in a ratio of about 10:1, i.e. wherein the culture comprises about 90% feeder cells.

The feeder cells according to the present invention may be unmodified autologous or allogeneic non-γδ T cells, i.e. they are cells derived from the same or different donor as the composition enriched for γδ T cells. Such feeder cells include αβ T cells and optionally Natural Killer cells (NK cells) derived from the same tissue or same tissue type (independently of being derived from either the same/a single or a different donor) as the composition enriched for γδ T cells. For example, wherein γδ T cells are isolated from non-haematopoietic tissue such as skin, the feeder cells may be non-γδ T cells also isolated from said non-haematopoietic tissue (e.g. skin). Such feeder cells, including αβ T cells may also be initially isolated from haematopoietic tissues but subsequently modified through cell culture or genetic manipulation to resemble the phenotype and biology of tissue resident or memory αβ T cells not normally found in haematopoietic tissues in large quantities. Thus, in one embodiment the feeder cells and the composition enriched for γδ T cells are derived from a single donor. In another embodiment, the feeder cells and the composition enriched for γδ T cells are derived from different donors.

In one embodiment, the composition of γδ T cells is derived from a single donor. In an alternative embodiment, the composition is derived from multiple donors, i.e. the composition is a ‘pooled’ composition. In a further embodiment, the feeder cells are derived from a single donor. In another embodiment, the feeder cells are derived from multiple donors, i.e. the feeder cells are ‘pooled’. Thus, in one embodiment, the feeder cells are obtained from multiple donors and the composition enriched for γδ T cells is obtained from a single donor. In another embodiment, the feeder cells are obtained from a single donor and the composition enriched for γδ T cells is obtained from multiple donors.

In one embodiment the single or multiple donors may comprise a subject which is to be treated with the cell populations or compositions of the invention. Alternatively, the single or multiple donors do not comprise a subject which is to be treated with the cell populations or compositions of the invention.

In some embodiments, the feeder cells comprise a population of αβ-rich T cells. In a further embodiment, the feeder cells comprise αβ T cells. In one embodiment, the αβ T cells comprise CD4 T cells and/or CD8 T cells. It will be understood that reference to “CD4 T cells” or “CD4⁺ T cells” refer to a type of T cell that expresses the CD4 surface protein. Equally, reference to “CD8 T cells” or “CD8⁺ T cells” refer to a type of T cell that expresses the CD8 surface protein. In a particular embodiment, the feeder cells comprise CD4 T cells. In a further embodiment, the feeder cells consist of CD4 T cells.

In a yet further embodiment, the feeder cells comprise a mixed population of αβ T cells and Natural Killer (NK) cells. Thus, in one embodiment the feeder cells additionally comprise Natural Killer (NK) cells.

It will be appreciated that the feeder cells described herein provide natural antigen presenting and co-stimulatory abilities, are not genetically modified to function as antigen presenting cells and are thus not aAPC. Furthermore, arresting the growth of the feeder cells, such as by irradiation or mitomycin-C treatment is not required because they are not derived from tumour cells. However, in another embodiment, the feeder cells are growth arrested. Methods of growth arrest are known in the art and include, without limitation, irradiation (e.g. γ-irradiation) and mitomycin-C treatment, yielding feeder cells which are unable to replicate but remain metabolically active, thus providing sufficient growth support to the γδ T cells. Arresting the growth of feeder cells enables the long-term culture of γδ T cells without the outgrowth of these cells when present in large numbers/a large proportion compared to the γδ T cells. Thus, in a further embodiment the feeder cells are irradiated. In an alternative embodiment, the feeder cells are mitomycin-C treated.

In one embodiment, the feeder cells are obtained from non-haematopoietic tissue. In a further embodiment, the feeder cells are obtained from skin. Examples of such non-haematopoietic tissue and methods for the preparation thereof may be found in WO2020095058 and, WO2020095059, the disclosures of which are incorporated in their entirety.

In other embodiments, the composition enriched for γδ T cells comprises NK cells. Thus, in one embodiment, step (i) comprises depletion of αβ T cells, i.e. the composition enriched for γδ T cells is prepared by depletion of αβ T cells. In a further embodiment, preparing a composition enriched for γδ T cells according to step (i) comprises depletion of αβ T cells from a mixed cell population obtained from a starting sample, such as non-haematological tissue as described hereinbefore. The presence of NK cells in the composition is advantageous as these cells are also effective cytotoxic cells. Therefore, a composition of γδ T cells additionally comprising NK cells may have enhanced cytotoxic properties compared to a composition of γδ T cells alone.

NK cells (also known as large granular lymphocytes (LGL)) are cytotoxic lymphocytes of the innate immune system. They provide rapid responses to e.g. virus-infected cells and tumour cells independently of MHC expression on the surface of the target cell. Therefore, similarly to γδ T cells, the recognition of target cells by NK cells is not MHC restricted and they are not allo-HLA reactive, meaning HLA matching of patients is not required for NK cell-based therapies.

Therefore, according to another aspect of the invention, there is provided a method for expanding γδ T cells, wherein said method comprises the steps of:
(i) preparing a composition enriched for γδ T cells by depletion of αβ T cells; and
(ii) culturing the composition in the presence of feeder cells, wherein the feeder cells are present in a ratio of at least 3:2 (feeder cells γδ T cells).

Thus, in certain embodiments the culture comprises at least 60% feeder cells. In other embodiments, the culture comprises at least 66% feeder cells, such as at least 70% feeder cells.

In another embodiment, step (i) comprises positive selection of γδ T cells from a mixed cell population obtained from a starting sample.

In certain embodiments, the starting sample is the starting sample is human tissue. In further embodiments, the starting sample is non-haematopoietic tissue, such as described hereinbefore. In a particular embodiment, the starting sample is skin.

In certain embodiments, the method comprises removing the feeder cells from the expanded γδ T cells by depletion of αβ T cells. Such removal by depletion of αβ T cells results in a population of expanded γδ T cells produced by the methods described herein which further comprises NK cells. As described hereinbefore, NK cells are good effector cells which, similarly to γδ T cells are nether MHC restricted nor allo-HLA reactive. Therefore, in a particular embodiment the population of expanded γδ T cells comprises NK cells. In an alternative embodiment, the method comprises removing the feeder cells from the expanded γδ T cells by positive selection of γδ T cells. Such positive selection of γδ T cells results in a highly purified population of γδ T cells which may be more appropriate for downstream processing or use in therapy compared to a population comprising other/additional cell types.

In one embodiment, the composition is cultured in media comprising IL-15. In a further embodiment, the composition is cultured in media comprising IL-21. Thus, in some embodiments the media comprises IL-15 and IL-21. In a yet further embodiment, the media additionally comprises IL-2. In a still further embodiment, the media additionally comprises IL-4. Thus, in some embodiments the media additionally comprises IL-2 and IL-4. In further embodiments, the media comprises IL-15, IL-21, IL-2 and IL-4.

In a particular embodiment, the composition enriched for γδ T cells is cultured in step (ii) in the presence of media comprising IL-15 and IL-21. In further embodiments, step (ii) comprises the conditions and/or methods for expanding γδ T cells disclosed in WO2017072367 and WO2018202808, the contents of which are incorporated in their entirety.

Therefore, according to another aspect of the invention, there is provided a method for expanding γδ T cells, wherein said method comprises the steps of:
(i) preparing a composition enriched for γδ T cells; and
(ii) culturing the composition in the presence of feeder cells and media comprising IL-15 and IL-21, wherein the feeder cells are present in a ratio of at least 3:2 (feeder cells: γδ T cells).

As used herein, “IL-15” refers to native or recombinant IL-15 or a variant thereof that acts as an agonist for one or more IL-15 receptor (IL-15R) subunits (e.g. mutants, muteins, analogues, subunits, receptor complexes, fragments, isoforms, and peptidomimetics thereof). IL-15, like IL-2, is a known T-cell growth factor that can support proliferation of an IL-2-dependent cell line, CTLL-2. IL-15 was first reported by Grabstein, et al. (Grabstein, et al. Science 1994. 264.5161: 965-969) as a 114-amino acid mature protein. The term “IL-15,” as used herein, means native or recombinant IL-15 and muteins, analogs, subunits thereof, or complexes thereof (e.g. receptor complexes, e.g. sushi peptides, as described in WO 2007/046006), and each of which can stimulate proliferation of CTLL-2 cells. In the CTLL-2 proliferation assays, supernatants of cells transfected with recombinantly expressed precursor and in-frame fusions of mature forms of IL-15 can induce CTLL-2 cell proliferation.

Human IL-15 can be obtained according to the procedures described by Grabstein, et al. or by conventional procedures such as polymerase chain reaction (PCR). A deposit of human IL-15 cDNA was made with the ATCC® on Feb. 19, 1993 and assigned accession number 69245.

The amino acid sequence of human IL-15 (Gene ID 3600) is found in Genbank under accession locator NP000576.1 GI: 10835153 (isoform 1) and NP_751915.1 GI: 26787986 (isoform 2). The murine (Mus musculus) IL-15 amino acid sequence (Gene ID 16168) is found in Genbank under accession locator NP_001241676.1 GI: 363000984.

IL-15 can also refer to IL-15 derived from a variety of mammalian species, including, for example, human, simian, bovine, porcine, equine, and murine. An IL-15 “mutein” or “variant”, as referred to herein, is a polypeptide substantially homologous to a sequence of a native mammalian IL-15 but that has an amino acid sequence different from a native mammalian IL-15 polypeptide because of an amino acid deletion, insertion or substitution. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gin and Asn. Other such conservative substitutions, for example, substitutions of entire regions having similar hydrophobicity characteristics, are well known. Naturally occurring IL-15 variants are also encompassed by the invention. Examples of such variants are proteins that result from alternate mRNA splicing events or from proteolytic cleavage of the IL-15 protein, wherein the IL-15 binding property is retained. Alternate splicing of mRNA may yield a truncated but biologically active IL-15 protein. Variations attributable to proteolysis include, for example, differences in the N- or C-termini upon expression in different types of host cells, due to proteolytic removal of one or more terminal amino acids from the IL-15 protein (generally from 1-10 amino acids). In some embodiments, the terminus of the protein can be modified to alter its physical properties, for example, with a chemical group such as polyethylene glycol (Yang, et al. Cancer 1995. 76:687-694). In some embodiments, the terminus or interior of the protein can be modified with additional amino acids (Clark-Lewis, et al. PNAS 1993. 90:3574-3577).

In some embodiments, the methods defined herein include IL-15 typically at a concentration of at least 0.1 ng/ml, such as at least 10 ng/ml (e.g. from 0.1 ng/ml to 10,000 ng/ml, from 1.0 ng/ml to 1,000 ng/ml, from 5 ng/ml to 800 ng/ml, from 10 ng/ml to 750 ng/ml, from 20 ng/ml to 500 ng/ml, from 50 ng/ml to 400 ng/ml, or from 100 ng/ml to 250 ng/mL, e.g. from 0.1 ng/ml to 1.0 ng/ml, from 1.0 ng/ml to 5.0 ng/ml, from 5.0 ng/ml to 10 ng/ml, from 10 ng/ml to 20 ng/ml, from 20 ng/ml to 100 ng/ml, from 20 ng/mL to 50 ng/ml, from 40 ng/ml to 70 ng/ml, from 50 ng/ml to 100 ng/ml, from 50 ng/ml to 60 ng/ml, from 100 ng/ml to 200 ng/mL, from 200 ng/ml to 500 ng/ml, or from 500 ng/ml to 1,000 ng/ml). In further embodiments, the methods defined herein include IL-15 typically at a concentration of less than 500 ng/ml, such as less 100 ng/ml. In some embodiments, the concentration of IL-15 is about 50 ng/ml. In another embodiment, the concentration of IL-15 is about 55 ng/ml.

As used herein, “IL-21” refers to native or recombinant IL-21 or a variant thereof that acts as an agonist for one or more IL-21 receptor (IL-21R) subunits (e.g. mutants, muteins, analogues, subunits, receptor complexes, fragments, isoforms, and peptidomimetics thereof). Such agents can support proliferation of natural killer (NK) and cytotoxic (CD8⁺) T cells. Mature human IL-21 occurs as a 133 amino acid sequence (less the signal peptide, consisting of an additional 22 N-terminal amino acids). An IL-21 mutein is a polypeptide wherein specific substitutions to the Interleukin-21 protein have been made while retaining the ability to bind IL-21Ra, such as those described in U.S. Pat. No. 9,388,241. The IL-21 muteins can be characterized by amino acid insertions, deletions, substitutions and modifications at one or more sites in or at the other residues of the native IL-21 polypeptide chain. In accordance with this disclosure any such insertions, deletions, substitutions and modifications result in an IL-21 mutein that retains the IL-21R binding activity. Exemplary muteins can include substitutions of 1, 2, 3, 4, 5, 6, 7, 8, 9, 10 or more amino acids.

Nucleic acid encoding human IL-21 can be obtained by conventional procedures such as polymerase chain reaction (PCR). The amino acid sequence of human IL-21 (Gene ID 59067) is found in Genbank under accession locator NC_000004.12. The murine (Mus musculus) IL-21 amino acid sequence (Gene ID 60505) is found in Genbank under accession locator NC_000069.6.

IL-21 can also refer to IL-21 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gin and Asn. Other such conservative substitutions, for example, substitutions of entire regions having similar hydrophobicity characteristics, are well known. Naturally occurring IL-21 variants are also encompassed by the invention. Examples of such variants are proteins that result from alternate mRNA splicing events or from proteolytic cleavage of the IL-21 protein, wherein the IL-21 binding property is retained. Alternate splicing of mRNA may yield a truncated but biologically active IL-21 protein. Variations attributable to proteolysis include, for example, differences in the N- or C-termini upon expression in different types of host cells, due to proteolytic removal of one or more terminal amino acids from the IL-21 protein (generally from 1-10 amino acids). In some embodiments, the terminus of the protein can be modified to alter its physical properties, for example, with a chemical group such as polyethylene glycol (Yang, et al. Cancer 1995. 76:687-694). In some embodiments, the terminus or interior of the protein can be modified with additional amino acids (Clark-Lewis, et al. PNAS 1993. 90:3574-3577).

In further embodiments, the methods defined herein include IL-21 typically at a concentration of at least 0.1 ng/ml, such as at least 1.0 ng/mL (e.g. from 0.1 ng/ml to 1,000 ng/ml, from 1.0 ng/mL to 100 ng/ml, from 1.0 ng/ml to 50 ng/ml, from 2 ng/ml to 50 ng/ml, from 3 ng/ml to 10 ng/ml, from 4 ng/ml to 8 ng/ml, from 5 ng/ml to 10 ng/ml, from 6 ng/ml to 8 ng/ml, e.g. from 0.1 ng/ml to 10 ng/ml, from 1.0 ng/ml to 5 ng/ml, from 1.0 ng/mL to 10 ng/ml, from 1.0 ng/ml to 20 ng/ml). In further embodiments, the methods defined herein include IL-21 typically at a concentration of less than 100 ng/ml, such as less 50 ng/ml. In some embodiments, the concentration of IL-21 is about 6 ng/ml, such as about 6.25 ng/mL.

As used herein, “IL-2” refers to native or recombinant IL-2 or a variant thereof that acts as an agonist for one or more IL-2 receptor (IL-2R) subunits (e.g. mutants, muteins, analogues, subunits, receptor complexes, fragments, isoforms, and peptidomimetics thereof). Such agents can support proliferation of an IL-2-dependent cell line, CTLL-2 (33; American Type Culture Collection (ATCC®) TIB 214). Mature human IL-2 occurs as a 133 amino acid sequence (less the signal peptide, consisting of an additional 20 N-terminal amino acids), as described in Fujita, et al. Cell 1986. 46.3:401-407. An IL-2 mutein is a polypeptide wherein specific substitutions to the Interleukin-2 protein have been made while retaining the ability to bind IL-2Rβ, such as those described in US 2014/0046026. The IL-2 muteins can be characterized by amino acid insertions, deletions, substitutions and modifications at one or more sites in or at the other residues of the native IL-2 polypeptide chain. In accordance with this disclosure any such insertions, deletions, substitutions and modifications result in an IL-2 mutein that retains the IL-2Rβ binding activity. Exemplary muteins can include substitutions of 1, 2, 3, 4, 5, 6, 7, 8, 9, 10 or more amino acids.

Nucleic acid encoding human IL-2 can be obtained by conventional procedures such as polymerase chain reaction (PCR). The amino acid sequence of human IL-2 (Gene ID 3558) is found in Genbank under accession locator NP_000577.2 GI: 28178861. The murine (Mus musculus) IL-2 amino acid sequence (Gene ID 16183) is found in Genbank under accession locator NP_032392.1 GI: 7110653.

IL-2 can also refer to IL-2 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gin and Asn. Other such conservative substitutions, for example, substitutions of entire regions having similar hydrophobicity characteristics, are well known. Naturally occurring IL-2 variants are also encompassed by the invention. Examples of such variants are proteins that result from alternate mRNA splicing events or from proteolytic cleavage of the IL-2 protein, wherein the IL-2 binding property is retained. Alternate splicing of mRNA may yield a truncated but biologically active IL-2 protein. Variations attributable to proteolysis include, for example, differences in the N- or C-termini upon expression in different types of host cells, due to proteolytic removal of one or more terminal amino acids from the IL-2 protein (generally from 1-10 amino acids). In some embodiments, the terminus or interior of the protein can be modified to alter its physical properties, for example, with a chemical group such as polyethylene glycol (Yang, et al. Cancer 1995. 76: 687-694). In some embodiments, the terminus or interior of the protein can be modified with additional amino acids (Clark-Lewis, et al. PNAS 1993. 90:3574-3577).

In certain embodiments, the methods defined herein include IL-2 typically at a concentration of at least 10 IU/mL, such as at least 100 IU/mL (e.g. from 10 IU/mL to 1,000 IU/mL, from 20 IU/mL to 800 IU/mL, from 25 IU/mL to 750 IU/mL, from 30 IU/mL to 700 IU/mL, from 40 IU/mL to 600 IU/mL, from 50 IU/mL to 500 IU/mL, from 75 IU/mL to 250 IU/mL, or from 100 IU/mL to 200 IU/mL, e.g. from 10 IU/mL to 20 IU/mL, from 20 IU/mL to 30 IU/mL, from 30 IU/mL to 40 IU/mL, from 40 IU/mL to 50 IU/mL, from 50 IU/mL to 75 IU/mL, from 75 IU/mL to 100 IU/mL, from 100 IU/mL to 150 IU/mL, from 150 IU/mL to 200 IU/mL, from 200 IU/mL to 500 IU/mL, or from 500 IU/mL to 1,000 IU/mL). In certain embodiments, the methods defined herein include IL-2 typically at a concentration of less than 1,000 IU/mL, such as less than 500 IU/mL. In some embodiments, the concentration of IL-2 is about 100 IU/mL.

As used herein, “IL-4” refers to native or recombinant IL-4 or a variant thereof that acts as an agonist for one or more IL-4 receptor (IL-4R) subunits (e.g. mutants, muteins, analogues, subunits, receptor complexes, fragments, isoforms, and peptidomimetics thereof). Such agents can support differentiation of naïve helper T cells (Th0 cells) to Th2 cells. Mature human IL-4 occurs as a 129 amino acid sequence (less the signal peptide, consisting of an additional 24 N-terminal amino acids). An IL-4 mutein is a polypeptide wherein specific substitutions to the Interleukin-4 protein have been made while retaining the ability to bind IL-4Rα, such as those described in U.S. Pat. No. 6,313,272. The IL-4 muteins can be characterized by amino acid insertions, deletions, substitutions and modifications at one or more sites in or at the other residues of the native IL-4 polypeptide chain. In accordance with this disclosure any such insertions, deletions, substitutions and modifications result in an IL-4 mutein that retains the IL-2Ra binding activity. Exemplary muteins can include substitutions of 1, 2, 3, 4, 5, 6, 7, 8, 9, 10 or more amino acids.

Nucleic acid encoding human IL-4 can be obtained by conventional procedures such as polymerase chain reaction (PCR). The amino acid sequence of human IL-4 (Gene ID 3565) is found in Genbank under accession locator NG_023252. The murine (Mus musculus) IL-4 amino acid sequence (Gene ID 16189) is found in Genbank under accession locator NC_000077.6.

IL-4 can also refer to IL-4 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gin and Asn. Other such conservative substitutions, for example, substitutions of entire regions having similar hydrophobicity characteristics, are well known. Naturally occurring IL-4 variants are also encompassed by the invention. Examples of such variants are proteins that result from alternate mRNA splicing events or from proteolytic cleavage of the IL-4 protein, wherein the IL-4 binding property is retained. Alternate splicing of mRNA may yield a truncated but biologically active IL-4 protein. Variations attributable to proteolysis include, for example, differences in the N- or C-termini upon expression in different types of host cells, due to proteolytic removal of one or more terminal amino acids from the IL-4 protein (generally from 1-10 amino acids). In some embodiments, the terminus of the protein can be modified to alter its physical properties, for example, with a chemical group such as polyethylene glycol (Yang, et al. Cancer 1995. 76:687-694). In some embodiments, the terminus or interior of the protein can be modified with additional amino acids (Clark-Lewis, et al. PNAS 1993. 90:3574-3577).

In further embodiments, the methods defined herein include IL-4 typically at a concentration of at least 0.1 ng/ml, such as at least 10 ng/ml (e.g. from 0.1 ng/ml to 1,000 ng/mL, from 1.0 ng/ml to 100 ng/ml, from 1.0 ng/mL to 50 ng/ml, from 2 ng/ml to 50 ng/ml, from 3 ng/ml to ng/ml, from 4 ng/mL to 30 ng/ml, from 5 ng/ml to 20 ng/mL, from 10 ng/ml to 20 ng/ml, e.g. from 0.1 ng/ml to 50 ng/ml, from 1.0 ng/mL to 25 ng/ml, from 5 ng/ml to 25 ng/ml). In further embodiments, the methods defined herein include IL-4 typically at a concentration of less than 100 ng/ml, such as less 50 ng/ml, in particular less than 20 ng/ml. In some embodiments, the concentration of IL-4 is about 15 ng/mL.

The γδ T cells described herein may also be gene engineered for enhanced therapeutic properties, such as for CAR-T therapy. This involves the generation of engineered cell receptors, such as chimeric antigen receptors (CARs) or engineered T cell receptors (TCRs), to re-program the T cell with a new specificity, e.g. the specificity of a monoclonal antibody. The engineered CAR or TCR may make the T cells specific for malignant cells and therefore useful for cancer immunotherapy. For example, the T cells may recognise cancer cells expressing a tumour antigen, such as a tumour specific antigen that is not expressed by normal somatic cells from the subject tissue, a tumour associated antigen which is preferentially overexpressed on cancer cells compared to healthy somatic cells or antigens expressed in the context of stress events such as oxidative stress, DNA damage, UV radiation, EGF receptor stimulation; or other means for identifying cancerous versus noncancerous cells. Thus, the CAR-modified T cells may be used for adoptive T cell therapy of, for example, cancer patients.

Therefore, in one embodiment, the methods described herein comprise transducing the composition of γδ T cells to express a surface receptor of interest, such as a chimeric antigen receptor (CAR) recognizing a tumour antigen. Any such CAR may be used in the present invention, including CARs targeting CD19 or other known tumour associated antigens.

The use of blood-resident γδ T cells for CAR-T therapy has been described. However, non-haematopoietic γδ T cells obtained by the method of the invention are likely to be particularly good vehicles for CAR-T approaches, as they can be transduced with chimeric antigen-specific receptors while retaining their innate-like capabilities of recognising transformed cells and are likely to have better tumour penetration and retention capabilities than either blood-resident γδ T cells or conventional, systemic αβ T cells. Furthermore, their lack of MHC dependent antigen presentation reduces the potential for graft-versus-host disease and permits them to target tumours expressing low levels of MHC. Likewise, their non-reliance upon conventional co-stimulation, for example via engagement of CD28, enhances the targeting of tumours expressing low levels of ligands for co-stimulatory receptors.

According to a further aspect of the invention, there is provided a method for engineering γδ T cells, said method comprising the steps of:
(i) preparing a composition enriched for γδ T cells;
(ii) transducing the composition to express a chimeric antigen receptor (CAR) recognizing a tumour antigen; and
(iii) culturing the transduced composition to expand the engineered γδ T cells, wherein steps (ii) and (iii) may be performed in either order or concurrently.

In one embodiment, step (ii) is performed prior to step (iii). Thus, according to this embodiment transduction of the composition is performed in the absence of any feeder cells which may be present in the culture. Therefore, the amount of material used for transduction may be reduced due to only the γδ T cells being transduced. In an alternative embodiment, step (ii) is performed concurrently with step (iii). According to this embodiment, transduction of the composition is performed in the presence of any feeder cells in the culture. Therefore, while the amount of transduction material may need to be increased compared to wherein step (ii) is performed prior to step (iii), it will be appreciated that handling may be reduced leading to a simpler overall method and reduced losses which may be associated with said handling.

Thus, in some embodiments step (iii) comprises culturing the transduced composition in the presence of feeder cells. In further embodiments, the method according to this aspect comprises any of the steps described hereinbefore.

It has been surprisingly found that the composition enriched for γδ T cells, particularly γδ T cells derived from non-haematopoietic tissue, does not require TCR (T cell receptor) stimulation, unlike previously known methods of T cell transduction, including γδ T cell transduction which require TCR stimulation by, e.g. an anti-CD3 antibody such as OKT-3, or an anti-γδ TCR antibody, such as an anti-Vδ1 antibody. Therefore, the methods described herein comprise transducing the composition of γδ T cells in the absence of TCR stimulation.

In certain embodiments, the composition is transduced using a viral vector. Such viral vectors are known in the art and the skilled person will be able to recognise the appropriate viral vector to be used according to the cells to be transduced. In one embodiment, the viral vector is a lentiviral vector or a retroviral vector, such as a gammaretroviral vector. In a further embodiment, the viral vector is a gammaretroviral vector, such as murine stem cell virus (MSCV) or Moloney Murine Leukemia Virus (MLV). In a yet further embodiment, the viral vector is pseudotyped with an envelope other than vesicular stomatitis virus-G (VSV-G), for example a betaretroviral envelope such as baboon endogenous virus (BaEV) or RD114.

In some embodiments, step (ii) is performed using between 1×10⁶ and 1×10⁸ TU/ml, such as about 1×10⁶, about 5×10⁶, about 1×10⁷, about 5×10⁷ or about 1×10⁸ TU/ml of viral vector. In a particular embodiment, step (ii) is performed using 1×10⁷ TU/ml of viral vector. In other embodiments, step (ii) is performed using an MOI of viral vector between 0.5 and 50, such as an MOI of about 0.5, about 1, about 1.5, about 2.5, about 5, about 10, about 25, about 40 or about 50. In one embodiment, step (ii) is performed using an MOI of viral vector of 2.5. In another embodiment, step (ii) is performed using an MOI of viral vector of 5. In a further embodiment, step (ii) is performed using an MOI of viral vector of 10.

In one embodiment, the tumour associated antigen is an antigen associated with a solid tumour. Thus, in some embodiments the tumour and/or cancer is a solid tumour. Constitutive expression of CD70, a member of the tumour necrosis family, has been described in both haematological and solid cancers where it increases the survival of tumour cells and regulatory T cells within the tumour microenvironment by signalling through its receptor, CD27. Thus, in a further embodiment the solid tumour is a CD70⁺ tumour. It will be appreciated that CD70 may be used to target engineered γδ T cells to said tumours. Therefore, in a yet further embodiment the tumour associated antigen is CD70.

In an alternative embodiment, the tumour associated antigen is mesothelin. Mesothelin is a kDa protein that is expressed in mesothelial cells and is overexpressed in several tumours, including mesothelioma, ovarian cancer, pancreatic adenocarcinoma, lung adenocarcinoma and cholangiocarcinoma. It has therefore been proposed as a tumour marker or tumour associated antigen which may be targeted in immunotherapy (Hassan et al. Clin. Cancer Res., 2004, 10(12):3937-3942). The expression of mesothelin in these tumours may contribute to the implantation and peritoneal spread of tumours by cell adhesion (Rump et al., Biological Chemistry, 2004, 279(10):9190-9198).

According to one aspect of the invention, there is provided an expanded γδ T cell population obtained by the methods described herein. According to a further aspect, there is provided an engineered γδ T cell population obtained by the methods described herein.

In some embodiments, the expanded/engineered γδ T cell population comprises greater than 50% γδ T cells, such as greater that 75% γδ T cells, in particular greater that 85% γδ T cells. In one embodiment, the expanded/engineered population comprises Vδ1 cells, wherein less than 50%, such as less than 25% of the Vo1 cells express TIGIT. In one embodiment, the expanded/engineered population comprises Vδ1 cells, wherein more than 50%, such as more than 60% of the Vδ1 cells express CD27.

The expanded/engineered γδ T cell population obtained by the methods described herein may be used as a medicament, for example for adoptive T cell therapy. This involves the transfer of an expanded/engineered population obtained by the methods into a patient. The therapy may be autologous, i.e. the γδ T cells may be transferred back into the same patient from which they were obtained, or the therapy may be allogeneic, i.e. the γδ T cells from one person may be transferred into a different patient. In instances involving allogeneic transfer, the expanded/engineered population may be substantially free of αβ T cells. For example, αβ T cells may be depleted from the expanded/engineered population, e.g. after engineering, using any suitable means known in the art (e.g. by negative selection, e.g. using magnetic beads). A method of treatment may include: providing a sample of non-haematopoietic tissue obtained from a donor individual; expanding and/or engineering the γδ T cells as described herein to produce an expanded/engineered population; and administering the expanded/engineered population of γδ T cells to a recipient individual.

The patient or subject to be treated is preferably a human cancer patient (e.g. a human cancer patient being treated for a solid tumour) or a virus-infected patient (e.g. a CMV-infected or HIV infected patient). In some instances, the patient has and/or is being treated for a solid tumour. Because they are normally resident in non-haematopoietic tissues, tissue-resident Vδ1 T cells are also more likely to home to and be retained within tumour masses than their systemic blood-resident counterparts and adoptive transfer of these cells is likely to be more effective at targeting solid tumours and potentially other non-haematopoietic tissue-associated immunopathologies.

As γδ T cells are non-MHC restricted, they do not recognise a host into which they are transferred as foreign, which means that they are less likely to cause graft-versus-host disease. This means that they can be used “off the shelf” and transferred into any recipient, e.g. for allogeneic adoptive T cell therapy.

γδ T cells obtained by methods described herein express NKG2D and respond to a NKG2D ligand (e.g. MICA), which is strongly associated with malignancy. They also express a cytotoxic profile in the absence of any activation and are therefore likely to be effective at killing tumour cells. For example, the expanded/engineered γδ T cells obtained as described herein may express one or more, preferably all of IFN-γ, TNF-α, GM-CSF, CCL4, IL-13, Granulysin, Granzyme A and B, and Perforin in the absence of any activation. IL-17A may not be expressed.

The findings reported herein therefore provide compelling evidence for the practicality and suitability for the clinical application of the expanded/engineered γδ T cells obtained by the methods described herein as an “off-the-shelf” immunotherapeutic reagent. These cells possess innate-like killing, have no MHC restriction and display improved homing to and/or retention within tumours than do other T cells.

In some embodiments, a method of treatment of an individual with a solid tumour in a non-haematopoietic tissue may include: expanding/engineering γδ T cells from a sample from the individual as described herein to produce an expanded/engineered population; and administering the expanded/engineered population of γδ T cells to the individual. In alternative embodiments, the method of treatment comprises expanding/engineering γδ T cells from a sample from a different individual as described herein to produce an expanded/engineered population; and administering the expanded/engineered population of γδ T cells to the individual with a solid tumour. In one embodiment, the amount of expanded/engineered γδ T cells administered to the individual is a therapeutically effective amount.

In further embodiments, the method of treatment and/or the therapeutically effective amount comprises those disclosed in WO2020095058, the contents of which is incorporated in its entirety.

Pharmaceutical compositions may include expanded and/or engineered γδ T cells as described herein in combination with one or more pharmaceutically or physiologically acceptable carrier, diluents, or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g. aluminium hydroxide); and preservatives. Cryopreservation solutions which may be used in the pharmaceutical compositions of the invention include, for example, DMSO. Compositions can be formulated, e.g. for intravenous administration.

Thus, according to another aspect of the invention, there is provided a pharmaceutical composition comprising the expanded γδ T cell population or the engineered γδ T cell population as described herein.

In one embodiment, the pharmaceutical composition is substantially free of (e.g. there are no) detectable levels of a contaminant, e.g. endotoxin or mycoplasma.

According to a yet further aspect of the invention, there is provided the expanded γδ T cell population, the engineered γδ T cell population or the pharmaceutical composition as described herein for use as a medicament. In another aspect, there is provided the expanded γδ T cell population, the engineered γδ T cell population or the pharmaceutical composition as described herein for use in the treatment of cancer.

It will be understood that all embodiments described herein may be applied to all aspects of the invention.

As used herein, the term “about” includes up to and including 10% greater and up to and including 10% lower than the value specified, suitably up to and including 5% greater and up to and including 5% lower than the value specified, especially the value specified. The term “between” includes the values of the specified boundaries.

Certain aspects and embodiments of the invention will now be illustrated by way of the following examples and with reference to the figures described above.

### EXAMPLES

### Example 1: Expansion of Skin Derived γδ Cells Using Feeder Cells

Skin-resident cells were isolated as previously described in WO2020095058 and, WO2020095059. Skin-resident lymphocytes were defrosted and immediately processed to remove αβ T cell feeder cells to yield cultures enriched for γδ T cells. αβ depleted cultures were then expanded in the presence of an irradiated feeder cell population. Irradiated feeder cells from various backgrounds were trialled in this experiment; allogeneic peripheral blood lymphocytes (PBLs), allogeneic peripheral blood mononuclear cells (PBMCs), anti-CD3 CD28 activated allogeneic PBMCs (Act PBMCs) or allogeneic skin isolation cultures. Cocultures were then incubated for 7 days before harvest and flow analysis for lineage markers and Ki67 nuclear expression. The expression level of intranuclear Ki67 within γδ T cells was measured as well as the total number of Vo1 γδ T cells per well. Both the γδ T cell intracellular Ki67 expression and the overall number of Vo1 γδ T cells was highest in cultures stimulated with irradiated skin isolation cells as feeder cells, indicating γδ T cell proliferation. These results demonstrate the superiority of skin-resident lymphocytes over blood-based leukocytes as a feeder cell component in driving skin-derived γδ T cell proliferation. (FIGS. 1A-B)

In separate experiments, skin-resident lymphocytes were defrosted and immediately processed through 2 different selection strategies to yield γδ T cell enriched, αβ T cell depleted cultures. γδ T cell enrichment was performed thorough either positive selection of the γδ T cells (FIGS. 2A-B) or through γδ T cell negative selection enrichment by positively selecting out the αβ T cell fraction (FIGS. 3A-B). These cultures were then expanded either without the presence of αβ T cells as feeder cells, or as a set starting population expressed in % of non αβ T cells population in relation to autologous αβ T cell feeder cells (1%, 5%, 10%, 20% or 40% non-αβ cell content at DO with the remainder of the culture made up of autologous feeder cells). For positively selected γδ T cells, the negative fraction containing predominantly skin αβ T cells served as the feeder cell layer. In these experiments, feeder cell layers were not irradiated. For negatively selected γδ T cells, the positively selected αβ T cells served as the feeder cell layer. Cultures were subsequently expanded for 14 days in the presence of growth cytokines IL-15 and IL-21. Upon harvest at D14, the percentage of γδ T cells of the CD45 lymphocyte fraction, as well as the overall fold increase in γδ T cell growth from DO to D14, were recorded. The results clearly show increased γδ T cell fold-growth over the expansion period when feeder cells are present in culture. 21 day expansions were superior to 14 day expansions in terms of overall γδ T cell fold-growth in all cases. Both the negative and positive γδ T cell enrichment strategies on D0 resulted in successful expansions in both feeder cell and feeder cell free cultures.

### Example 2: Transduction of Skin Derived γδ Cells Using CD19 CAR

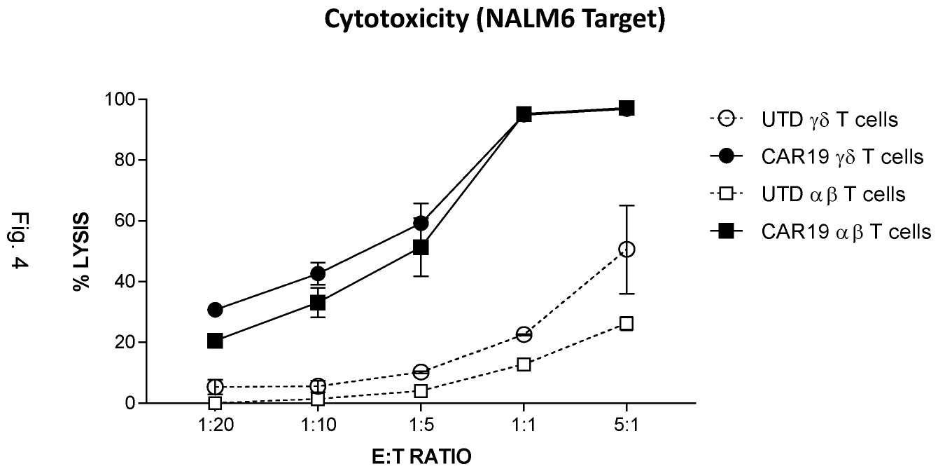

Skin-resident lymphocytes were defrosted and cultured for 7 days in the presence of IL-15 and IL-21. At day 7, all cells were harvested and transduced with vector encoding a CAR construct specific for CD19. Cells were then expanded for a further 7 days in the presence of IL-15 and IL-21 before harvest and cryopreservation. The transduction intervention did not affect the expansion of the skin-resident γδ T cells (data not shown). For functional assays, cryopreserved cells were defrosted and αβ T cells sorted via positive selection MACS processing, producing positively selected skin-resident αβ T cells and negatively selected skin-resident γδ T cells. γδ T cell or αβ T cell populations were cocultured alongside the haematological tumour cell line NALM6 at a variety of effector-target ratios. Cocultures were then incubated for 18 h and target cell lysis detected via SYTOX™ (Thermofisher) staining by flow cytometry. CAR Transduced skin-resident γδ T cells exhibited high functionality against the NALM6 cell line. This level of functionality was comparable to that of the donor matched CAR transduced skin αβ T cells. (FIG. 4)

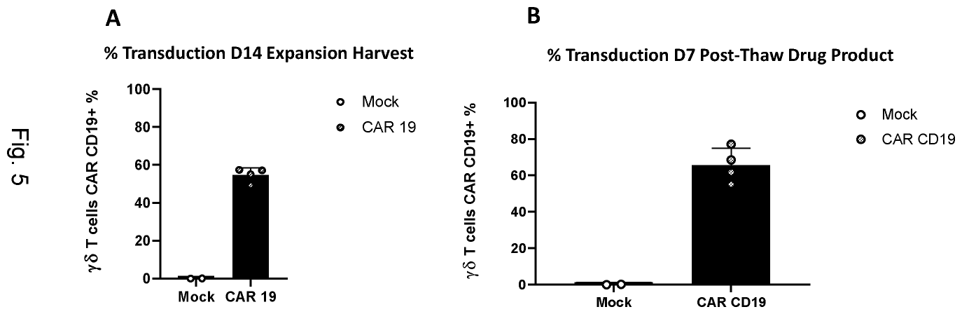

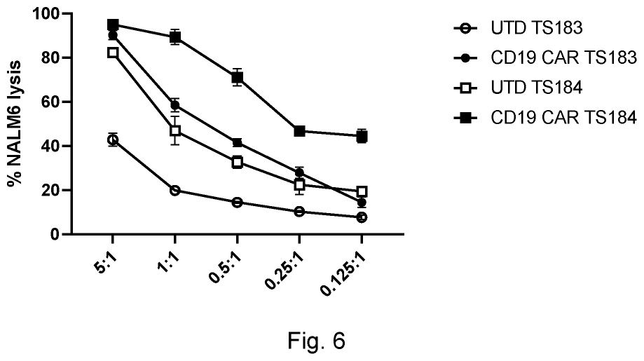

In separate experiments, skin-resident lymphocytes were defrosted and immediately processed to deplete αβ T cells via positive selection of αβ T cells via MACS. These αβ T cell depleted, γδ T cell enriched populations were cultured for 2 days in the presence of IL-15 and IL-21 prior to gene engineering. After 2 days, cultures were harvested and transduced with vector encoding a CD19-specific CAR construct. For 2 of the 4 donors, mock transduction cultures were established whereby cells underwent the same transduction protocol but without the presence of the vector. Post-transduction, cells were subsequently expanded for a further 12 days after which they were harvested, phenotyped via flow cytometry for lineage and CD19-specific CAR expression, and then cryopreserved. Results indicate that transduced γδ T cells express the CAR construct specific for CD19 while mock transduced controls (were applicable) did not (FIG. 5A). Furthermore, once cryopreserved cells were defrosted and cultured for a further 7 days in IL-15 and IL-21, the percentage of CAR⁺γδ T cells were stable (FIG. 5B). Cryopreserved cells were also used in functionality assays. Cells were defrosted and cocultured alongside the haematological tumour cell line NALM6 at a variety of effector: target ratios for 18 h. Results indicate that in the 2 donors tested, CAR transduced γδ T cells had improved cytotoxicity performance against NAML6 when compared to matched untransduced controls (FIG. 6).

### Example 3: Transduction and Expansion of Skin-Derived γδ Cells Using Mesothelin-CAR

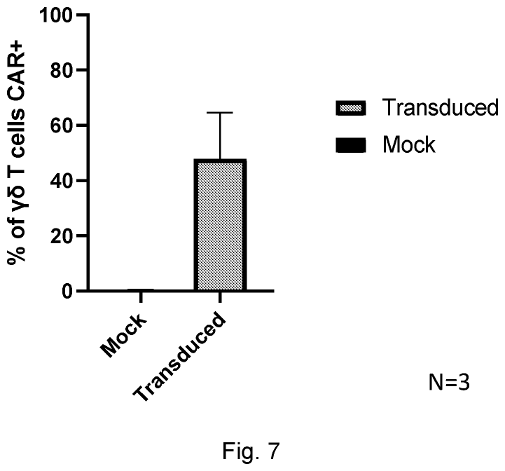

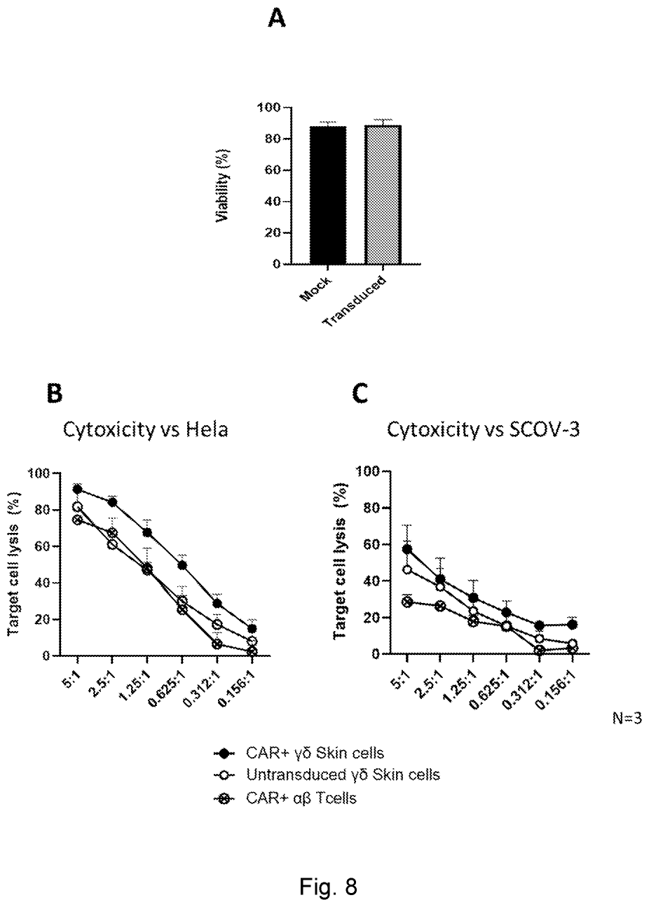

Skin-resident lymphocytes were defrosted and immediately processed to deplete αβ T cells via positive selection of αβ T cells via MACS. These αβ T cell depleted, γδ T cell enriched populations were cultured for 2 days in the presence of IL-15 and IL-21 prior to gene engineering. On day 2, cells were harvested from culture and transduced with RD-114 pseudotyped γ-retrovirus vector encoding a mesothelin-specific CAR construct. As a control, mock transduction cultures were established whereby cells underwent the same transduction protocol but without the presence of the vector. Cell were subsequently expanded for a further 12 days after which they were harvested, phenotyped via flow cytometry for lineage and CAR expression, and then cryopreserved. Transduced cells expressed the CAR construct while mock transduced controls did not (FIG. 7). Upon defrost, both mock and CAR transduced cells exhibited high viability (as measured via NC250 viable cell counting) (FIG. 8A).

Transduced and mock transduced cells were then defrosted and immediately tested for cytotoxicity against mesothelin-expressing solid tumour (adenocarcinoma) cell lines (Hela and SCOV-3). In addition to transduced γδ T cells, non-donor matched PBMC derived αβ T cells transduced with the same binder and expanded in IL-2 were also tested for cytotoxicity against the same solid tumour target cell lines. Cells were cultured at effector:target ratios of 5:1, 2.5:1, 1.25:1, 0.625:1, 0.312:1 and 0.156:1. Cytotoxicity co-cultures were incubated for 18 h hours before endpoint analysis. Cytotoxicity of solid tumour target cells was determined through enumeration of viable targets using the CellTitre GLO® (Promega) assay system. CAR transduced γδ T cells exhibited improved killing of both HeLa and SCOV-3 cell lines when compared to mock transduced controls (FIGS. 8B-C). Because untransduced γδ T cells have some activity against tumour cell lines, they display a similar cytotoxicity against the tumour cell lines as the CAR-transduced αβ T cells. However, the CAR-transduced γδ T cells show an increase in cytotoxicity as compared to untransduced γδ T cells and CAR-transduced αβ T cells.

### Example 4: Skin-Resident Cells were Isolated and Frozen as Described in Example 1

After thawing, γδ T cells were enriched through negative selection via magnetic activated cell sorting (MACS) and subsequently cocultured with a variety of different autologous positively selected αβ T cell populations, and the effect of coculture with αβ T cells upon γδ T cell expansion rate measured over 14 and 21 days of culture. Firstly γδ T cells were enriched from frozen isolated cells via depletion of αβ T cells via MACS. This resulted in populations of untouched (i.e., unlabelled with any magnetically labelled antibodies) γδ and TCR negative cells. These γδ T cell enriched populations were then cocultured with autologous CD4 αβ T cells (“CD4 Feeder”), CD8 αβ T cells (“CD8 Feeder”) or both CD4 and CD8 αβ T cells (“αβ Feeder”). All feeder cell layers were purified from skin resident cells via positive-labelling MACS selection. In all cocultures, cells were setup at a ratio of 10% γδ T cell enriched population with the remaining 90% of the culture made up of the autologous feeder cell layer, with cultures run in TexMACS media supplemented with 5% allogeneic plasma and 80 ng/ml IL-15 and 11.25 ng/ml IL-21. Cultures were then expanded for either 14 or 21 days and expansion of the γδ T cells in each culture setup recorded at each timepoint. Cultures were subject to a 48 h feeding regime of removal of 50% of media and replenishment with 50% media supplemented with cytokines sufficient to return the culture to the initial cytokine concentration. Feeder cells were not further added to cultures after DO setup. Control populations of γδ T cell enriched cultures expanded without the addition of any αβ feeder cells were established (“γδ only”).

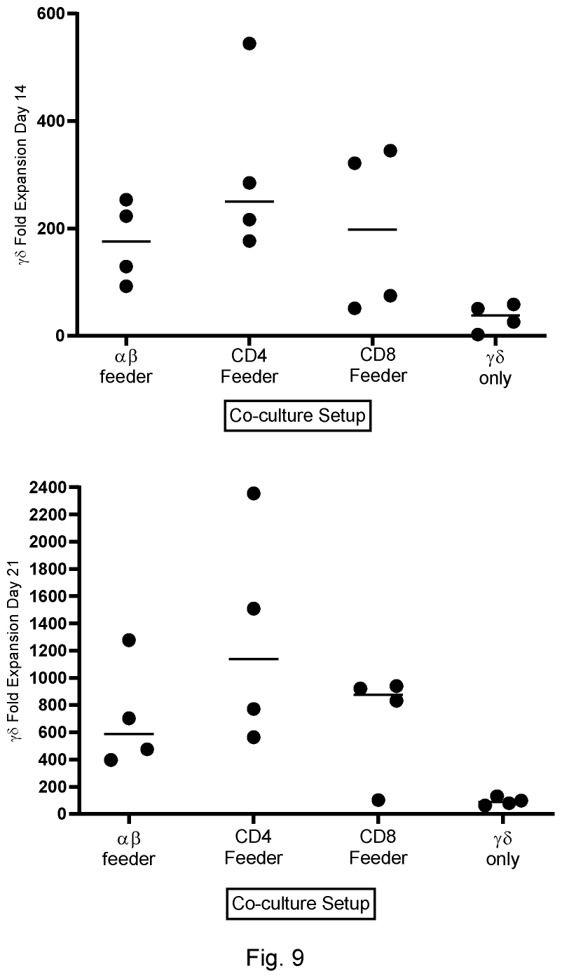

γδ T cell fold-expansion was boosted when co-cultured with any of the tested αβ T cell feeder cell cultures. Utilizing enriched CD4 αβ T cells provoked the greatest increase in γδ fold-expansion over both 14 and 21 days in culture. The results indicate that αβ T cells serve as an effective feeder cell layer to promote γδ T cell expansion, with CD4 αβ T cells being superior to CD8 αβ T cells in driving expansion (FIG. 9).

1. A method for expanding γδ T cells, wherein said method comprises the steps of:
(i) preparing a composition enriched for γδ T cells; and
(ii) culturing the composition in the presence of feeder cells, wherein the feeder cells are present in a ratio of at least 4:1 (feeder cells: γδ T cells).

2. A method for expanding γδ T cells, wherein said method comprises the steps of:
(i) preparing a composition enriched for γδ T cells; and
(ii) culturing the composition in the presence of feeder cells and media comprising IL-15 and IL-21, wherein the feeder cells are present in a ratio of at least 3:2 (feeder cells: γδ T cells).

3. A method for expanding γδ T cells, wherein said method comprises the steps of:
(i) preparing a composition enriched for γδ T cells by depletion of αβ T cells; and
(ii) culturing the composition in the presence of feeder cells, wherein the feeder cells are present in a ratio of at least 3:2 (feeder cells: γδ T cells).

4. The method according to any one of claims 1 to 3, wherein the feeder cells are present in a ratio of at least 4:1 (feeder cells: γδ T cells).

5. The method according to any one of claims 1 to 4, wherein the feeder cells are present in a ratio of at least 10:1 (feeder cells: γδ T cells).

6. The method according to any one of claims 1 to 5, wherein the feeder cells are present in a ratio of about 10:1 to about 99:1 (feeder cells: γδ T cells).

7. The method according to any one of claims 1 to 6, wherein the feeder cells comprise αβ T cells.

8. The method according to claim 7, wherein the αβ T cells comprise CD4 T cells.

9. The method according to claim 7 or claim 8, wherein the feeder cells additionally comprise Natural Killer (NK) cells.

10. The method according to any one of claims 1 to 9, wherein the feeder cells are irradiated.

11. The method according to any one of claims 1 to 10, wherein the feeder cells are derived from non-haematopoietic tissue.

12. The method according to claim 11, wherein the feeder cells are derived from skin.

13. The method according to any one of claims 1 to 12, wherein the feeder cells are derived from a single donor.

14. The method according to any one of claims 1 to 12, wherein the feeder cells are derived from multiple donors.

15. The method according to any one of claims 1 to 14, wherein the γδ T cells are derived from a single donor.

16. The method according to any one of claims 1 to 14, wherein the γδ T cells are derived from multiple donors.

17. The method according to any one of claims 1 to 16, wherein the feeder cells and the γδ T cells are derived from the same donor(s).

18. The method according to any one of claims 1 to 16, wherein the feeder cells and the γδ T cells are derived from different donor(s).

19. The method according to any one of claims 1 to 18, wherein the method comprises removing the feeder cells from the expanded γδ T cells by depletion of αβ T cells.

20. The method according to any one of claims 1 to 18, wherein the method comprises removing the feeder cells from the expanded γδ T cells by positive selection of γδ T cells.

21. A method for engineering γδ T cells, said method comprising the steps of:
(i) preparing a composition enriched for γδ T cells;
(ii) transducing the composition to express a chimeric antigen receptor (CAR) recognizing a tumour antigen in the absence of TCR stimulation; and
(iii) culturing the transduced composition to expand the engineered γδ T cells,
wherein steps (ii) and (iii) may be performed in either order or concurrently.

22. The method according to claim 21, wherein step (ii) is performed prior to step (iii).

23. The method according to claim 21, wherein step (ii) is performed concurrently with step (iii).

24. The method according to any one of claims 21 to 23, wherein the composition is transduced using a viral vector, such as a retroviral vector, such as a gammaretroviral vector or a lentiviral vector.

25. The method according to claim 24, wherein the viral vector is a gammaretroviral vector, such as murine stem cell virus (MSCV) or Moloney Murine Leukemia Virus (MLV).

26. The method according to claim 24 or claim 25, wherein the viral vector is pseudotyped with an envelope other than vesicular stomatitis virus-G (VSV-G), for example a betaretroviral envelope such as baboon endogenous virus (BaEV) or RD114.

27. The method according to any one of claims 24 to 26, wherein step (ii) is performed using 1×10⁷ TU/ml of viral vector.

28. The method according to any one of claims 21 to 27, wherein the tumour antigen is a tumour specific antigen that is not expressed by normal somatic cells from the subject tissue.

29. The method according to any one of claims 21 to 27, wherein the tumour antigen is a tumour associated antigen which is preferentially overexpressed on cancer cells compared to healthy somatic cells.

30. The method according to any one of claims 21 to 27, wherein the tumour antigen is an antigen expressed in the context of stress events such as oxidative stress, DNA damage, UV radiation, EGF receptor stimulation.

31. The method according to any one of claims 21 to 30, wherein the tumour antigen is an antigen associated with a solid tumour.

32. The method according to claim 31, wherein the solid tumour is a mesothelin⁺ tumour.

33. The method according to any one of claims 21 to 32, wherein the tumour associated antigen is mesothelin.

34. The method according to any one of claims 21 to 33, wherein step (iii) comprises culturing the transduced composition in the absence of feeder cells.

35. The method according to any one of claims 21 to 33, wherein step (iii) comprises culturing the transduced composition in the presence of feeder cells.

36. The method according to claim 32 comprising the steps of a method according to any one of claims 1 to 20.

37. The method according to any one of claims 1 to 36, wherein step (i) comprises depletion of αβ T cells from a mixed cell population obtained from a starting sample.

38. The method according to any one of claims 1 to 36, wherein step (i) comprises positive selection of γδ T cells from a mixed cell population obtained from a starting sample.

39. The method according to claim 37 or claim 38, wherein the starting sample is human tissue.

40. The method according to any one of claims 37 to 39, wherein the starting sample is non-haematopoietic tissue.

41. The method according to claim 40, wherein the starting sample is skin.

42. The method according to any one of claim 1 or 3 to 41, wherein the composition is cultured in media comprising IL-15 or IL-21.

43. The method according to claim 42, wherein the media comprises IL-15 and IL-21.

44. The method according to any one of claim 42 or 43, wherein the media additionally comprises IL-2 and/or IL-4

45. The method according to any one of claims 1 to 44, wherein the method comprises culturing the composition for between 7 and 21 days.

46. The method according to any one of claims 1 to 45, wherein the method comprises culturing the composition for about 10, 11, 12, 13, or 14 days.

47. The method according to any one of claims 1 to 42, wherein expanding the population of γδ T cells provides at least a 5-fold, especially at least a 10-fold, in particular at least a 20-fold, such as at least a 50-fold, for example at least a 100-fold number of γδ T cells.

48. The method according to any one of claims 1 to 47, wherein the method comprises freezing the expanded γδ T cells.

49. An expanded γδ T cell population obtainable, such as obtained, by the method of any one of claims 1 to 20 or 36 to 48.

50. An engineered γδ T cell population obtainable, such as obtained, by the method according to any one of claims 21 to 48.

51. A pharmaceutical composition comprising the expanded γδ T cell population according to claim 48 or engineered γδ T cell population according to claim 50.

52. The expanded γδ T cell population according to claim 49, the engineered γδ T cell population according to claim 50 or the pharmaceutical composition according to claim 51 for use as a medicament.

53. The expanded γδ T cell population according to claim 49, the engineered γδ T cell population according to claim 50 or the pharmaceutical composition according to claim 51 for use in the treatment of cancer.

54. The expanded γδ T cell population, engineered γδ T cell population or pharmaceutical composition for use according to claim 53, wherein the cancer is a solid tumour.
